(19) United States
(12) Patent Application Publication
(10) Pub. No.: US 2023/0348726 A1
(43) Pub. Date: Nov. 2, 2023

(54) CLOSED-CELL METAL OXIDE PARTICLES

(71) Applicant: BASF SE, Ludwigshafen Am Rhein (DE)

(72) Inventors: Liangliang QU, Tarrytown, NY (US); Paul A. ODORISIO, Tarrytown, NY (US); Michael David BURKE, Tarrytown, NY (US); Rupa Hiremath DARJI, Tarrytown, NY (US)

(21) Appl. No.: 18/014,915

(22) PCT Filed: Jul. 21, 2021
(86) PCT No.: PCT/IB2021/000499

§ 371 Date: Jan. 6, 2023

Related U.S. Application Data
(60) Provisional application No. 63/055,011, filed on Jul. 22, 2020.

(51) Int. Cl. C09C 1/00; C09C 3/04; C09C 3/10; C09C 3/00

(52) U.S. Cl. CPC C09C 1/0015; C09C 3/043; C09C 3/10; C09C 3/006; C01P 2006/14; C01P 2004/64; C09C 2200/505

(57) ABSTRACT

Disclosed in certain embodiments are closed-cell metal oxide particles and methods of preparing the same. In at least one embodiment, a closed-cell metal oxide particle comprises a metal oxide matrix defining an array of closed-cells. Each closed-cell encapsulates a media-inaccessible void volume. The outer surface of the closed-cell metal oxide particle is defined by the array of closed-cells.

### CROSS-REFERENCE TO RELATED APPLICATION(S)

This application claims the benefit of priority of U.S. Provisional Application No. 63/055,011, filed on Jul. 22, 2020, the disclosure of which is hereby incorporated by reference herein in its entirety.

### TECHNICAL FIELD

This application relates to metal oxide particles having, for example, structural colorant properties, as well as methods of preparing the same.

### BACKGROUND

Traditional pigments and dyes exhibit color via light absorption and reflection, relying on chemical structure. Structural colorants exhibit color via light interference effects, relying on physical structure as opposed to chemical structure. Structural colorants are found in nature, for example, in bird feathers, butterfly wings and certain gemstones. Structural colorants are materials containing micro- or nano-structured surfaces small enough to interfere with visible light and produce color. For example, such materials often contain nanoscale pore structures that contribute to their optical characteristics. However, media infiltration within exposed pores can impact these optical characteristics by changing the net refractive index or by changing the average refractive index within the pores.

### SUMMARY OF THE INVENTION

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method of preparing closed-cell metal oxide particles comprises: generating liquid droplets from a particle dispersion comprising first particles comprising a polymer material and second particles comprising a metal oxide material; drying the liquid droplets to provide dried particles comprising an array of the first particles; and calcining or sintering the dried particles. In at least one embodiment, each of the first particles is coated by a layer of the second particles. In at least one embodiment, calcining or sintering densifies the metal oxide material and removes the polymer material to produce the closed-cell metal oxide particles each comprising a metal oxide matrix defining an array of closed-cells, each closed-cell encapsulating a media-inaccessible void volume. In at least one embodiment, outer surfaces of the closed-cell metal oxide particles are defined by their respective arrays of closed-cells.

In at least one embodiment, the array of closed-cells is an ordered array. In at least one embodiment, the array of closed-cells is a disordered array.

In at least one embodiment, the first particles comprise net positive charged surfaces, and wherein the second particles comprise net negative charged surfaces. In at least one embodiment, the first particles comprise net negative charged surfaces, and wherein the second particles comprise net positive charged surfaces. In at least one embodiment, the surface charges drive the formation of the layer of the second particles on the first particles.

In at least one embodiment, the polymer material comprises a polymer selected from poly(meth)acrylic acid, poly(meth)acrylates, polystyrenes, polyacrylamides, polyethylene, polypropylene, polylactic acid, polyacrylonitrile, a co-polymer of methyl methacrylate and [2-(methacryloyloxy)ethyl]trimethylammonium chloride, derivatives thereof, salts thereof, copolymers thereof, or mixtures thereof.

In at least one embodiment, the first particles have an average diameter from about 50 nm to about 500 nm.

In at least one embodiment, the metal oxide material comprises a metal oxide selected from silica, titania, alumina, zirconia, ceria, iron oxides, zinc oxide, indium oxide, tin oxide, chromium oxide, and combinations thereof. In at least one embodiment, the metal oxide material comprises silica.

In at least one embodiment, the second particles have an average diameter from about 1 nm to about 120 nm.

In at least one embodiment, the closed-cell metal oxide particles have an average diameter from about 0.5 μm to about 100 μm.

In at least one embodiment, generating the liquid droplets is performed using a microfluidic process.

In at least one embodiment, generating and drying the liquid droplets is performed using a spray-drying process.

In at least one embodiment, generating the liquid droplets is performed using a vibrating nozzle.

In at least one embodiment, drying the droplets comprises evaporation, microwave irradiation, oven drying, drying under vacuum, drying in the presence of a desiccant, or a combination thereof.

In at least one embodiment, the particle dispersion is an aqueous particle dispersion.

In at least one embodiment, a weight to weight ratio of the first particles to the second particles is from about 1/10 to about 10/1.

In at least one embodiment, a weight to weight ratio of the first particles to the second particles is about 2/3, about 1/1, about 3/2, or about 3/1.

In at least one embodiment, a particle size ratio of the second particles to the first particles is from 1/50 to 1/5.

In another aspect of the present disclosure, a method of preparing closed-cell metal oxide particles comprises: generating liquid droplets from a particle dispersion comprising polymer in a sol-gel matrix of a metal oxide material, the polymer particles comprising a polymer material; drying the liquid droplets to provide dried particles comprising an array of the polymer particles; and calcining or sintering the dried particles to obtain the closed-cell metal oxide particles. In at least one embodiment, each of the polymer particles is coated by the sol-gel matrix. In at least one embodiment, the calcining or sintering removes the polymer material and densifies the metal oxide material to produce the closed-cell metal oxide particles each comprising a metal oxide matrix defining an array of closed-cells, each closed-cell encapsulating a media-inaccessible void volume. In at least one embodiment, outer surfaces of the closed-cell metal oxide particles are defined by their respective arrays of closed-cells.

In at least one embodiment, the polymer particles comprise net positive charged surfaces, and the sol-gel matrix of the metal oxide material comprises a net negative charge. In at least one embodiment, the polymer particles comprise net negative charged surfaces, and the sol-gel matrix of the metal oxide material comprises a net positive charge.

In another aspect of the present disclosure, closed-cell metal oxide particles are prepared by any of the aforementioned processes or any of the processes described herein.

In another aspect of the present disclosure, a closed-cell metal oxide particle comprises a metal oxide matrix defining an array of closed-cells, each closed-cell encapsulating a media-inaccessible void volume. In at least one embodiment, the outer surface of the closed-cell metal oxide particle is defined by the array of closed-cells.

In at least one embodiment, the array of closed-cells is an ordered array. In at least one embodiment, the array of closed-cells is a disordered array.

In at least one embodiment, the void volumes have an average diameter from about 50 nm to about 500 nm.

In at least one embodiment, the metal oxide matrix comprises a metal oxide selected from silica, titania, alumina, zirconia, ceria, iron oxides, zinc oxide, indium oxide, tin oxide, chromium oxide, and combinations thereof. In at least one embodiment, the metal oxide matrix comprises silica.

In at least one embodiment, the closed-cell metal oxide particle is derived at least partially from polymer particles having an average diameter from about 50 nm to about 500 nm. In at least one embodiment, the closed-cell metal oxide particle is derived at least partially from metal oxide particles having an average diameter from about 1 nm to about 120 nm.

In at least one embodiment, the closed-cell metal oxide particle is derived from a metal oxide precursor selected from silica, titania, alumina, zirconia, ceria, iron oxides, zinc oxide, indium oxide, tin oxide, chromium oxide, and combinations thereof.

In another aspect of the present disclosure, a composition comprises a plurality of the closed-cell metal oxide particle of any of the aforementioned embodiments or any of the embodiments described herein. In at least one embodiment, an average diameter of the closed-cell metal oxide particles range from about 0.5 μm to about 100 μm. In at least one embodiment, the composition further comprises a substrate having the closed-cell metal oxide particles disposed thereon.

In at least one embodiment, the closed-cell oxide particles of any of the embodiments described herein further comprise a light absorber. In at least one embodiment, the light absorber is present from 0.1 wt % to about 40.0 wt %. In at least one embodiment, the light absorber comprises carbon black. In at least one embodiment, the light absorber comprises one or more ionic species.

In another aspect of the present disclosure, a bulk composition exhibiting whiteness, a non-white color, or an effect in the ultraviolet spectrum, comprises a plurality of the closed-cell metal oxide particles according to any of the embodiments described herein.

Other aspects of the present disclosure are directed to compositions comprising the closed-cell metal oxide particles described herein in the form of an aqueous formulation, an oil-based formulation, an ink, a coating formulation, a food, a plastic, a cosmetic formulation, or a material for a medical application, or a security application.

As used herein, the term “bulk sample” refers to a population of particles. For example, a bulk sample of particles is simply a bulk population of particles, for example, ≥0.1 mg, ≥0.2 mg, ≥0.3 mg, ≥0.4 mg, ≥0.5 mg, ≥0.7 mg, ≥1.0 mg, ≥2.5 mg, ≥5.0 mg, ≥10.0 mg, or ≥25.0 mg. A bulk sample of particles may be substantially free of other components.

Also as used herein, the phrase “exhibits color observable by the human eye” means color will be observed by an average person. This may be for any bulk sample distributed over any surface area, for example, a bulk sample distributed over a surface area of from any of about 1 cm², about 2 cm², about 3 cm², about 4 cm², about 5 cm², or about 6 cm² to any of about 7 cm², about 8 cm², about 9 cm², about 10 cm², about 11 cm², about 12 cm², about 13 cm², about 14 cm², or about 15 cm². It may also mean observable by a CIE 1931 2° standard observer and/or by a CIE 1964 10° standard observer. The background for color observation may be any background, for example, a white background, black background, or a dark background anywhere between white and black.

Also as used herein, the term “of” may mean “comprising.” For example, “a liquid dispersion of” may be interpreted as “a liquid dispersion comprising.”

Also as used herein, the terms “particles,” “microspheres,” “microparticles,” “nanospheres,” “nanoparticles,” “droplets,” etc., may refer to, for example, a plurality thereof, a collection thereof, a population thereof, a sample thereof, or a bulk sample thereof.

Also as used herein, the terms “micro” or “micro-scaled,” for example, when referring to particles, mean from 1 micrometer (μm) to less than 1000 μm. The term “nano” or “nano-scaled,” for example, when referring to particles, mean from 1 nanometer (nm) to less than 1000 nm.

Also as used herein, the term “monodisperse” in reference to a population of particles means particles having generally uniform shapes and generally uniform diameters. A present monodisperse population of particles, for example, may have 90%, 91%, 92%, 93%, 94%, 95%, 96%, 97%, 98% m or 99% of the particles by number having diameters within ±7%, ±6%, ±5%, ±4%, ±3%, ±2%, or ±1% of the average diameter of the population.

Also as used herein, the term “media-inaccessible” in reference to a volume means that the volume is shielded from infiltration by large molecules (e.g., molecules, such as polymers and oligomers, having a molecular weight greater than 5000 g/mol). The volume may be accessible to solvents, such as water, toluene, hexane, and ethanol.

Also as used herein, the term “substantially free of other components” means containing, for example, ≤5%, ≤4%, ≤3%, ≤2%, ≤1%, ≤0.5%, ≤0.4%, ≤0.3%, ≤0.2%, or ≤0.1% by weight of other components.

The articles “a” and “an” used herein refer to one or to more than one (e.g., at least one) of the grammatical object. Any ranges cited herein are inclusive.

Also as used herein, the term “about” is used to describe and account for small fluctuations. For example, “about” may mean the numeric value may be modified by ±5%, ±4%, ±3%, ±2%, ±1%, ±0.5%, ±0.4%, ±0.3%, ±0.2%, ±0.1%, or ±0.05%. All numeric values are modified by the term “about” whether or not explicitly indicated. Numeric values modified by the term “about” include the specific identified value. For example, “about 5.0” includes 5.0.

Unless otherwise indicated, all parts and percentages are by weight. Weight percent (wt %), if not otherwise indicated, is based on an entire composition free of any volatiles, that is, based on dry solids content.

### BRIEF DESCRIPTION OF DRAWINGS

The disclosure described herein is illustrated by way of example and not by way of limitation in the accompanying figures.

FIG. 1A illustrates a metal oxide particle with a closed-cell morphology according to some embodiments of the present disclosure.

FIG. 1B illustrates a comparative metal oxide particle having a porous exterior surface.

FIG. 2 illustrates a process of preparing metal oxide particles having closed-cell morphologies according to some embodiments of the present disclosure.

FIG. 3 shows a schematic of an exemplary spray drying system used in accordance with various embodiments of the present disclosure.

FIG. 4 shows scanning electron microscope (SEM) images of closed-cell metal oxide particles produced via microfluidic technology according to embodiments of the present disclosure.

FIG. 5 shows photographs comparing closed-cell silica particles produced according to an embodiment of the present disclosure with porous particles to demonstrate the prevention of oil infiltration into voids of the closed-cell silica particles.

FIG. 6 shows SEM images of closed-cell silica particles produced via a spray drying process according to embodiments of the present disclosure.

FIG. 7 is a plot of the UV-vis spectrum for a sample produced according to an embodiment of the present disclosure, which shows a reflection peak at 440 nm corresponding to blue color.

FIG. 8 is a plot of the UV-vis spectrum for a sample produced according to an embodiment of the present disclosure, which shows a reflection peak at 520 nm corresponding to green color.

FIG. 9 is a plot of the UV-vis spectrum showing relative attenuation values in the UV range of closed-cell silica particles and silica nanoparticles produced according to embodiments of the present disclosure.

FIG. 10 shows SEM images of a closed-cell titania particle produced according to further embodiments of the present disclosure.

FIG. 11 shows an SEM image of a closed-cell silica particle produced via a sol-gel process according to embodiments of the present disclosure.

### DETAILED DESCRIPTION

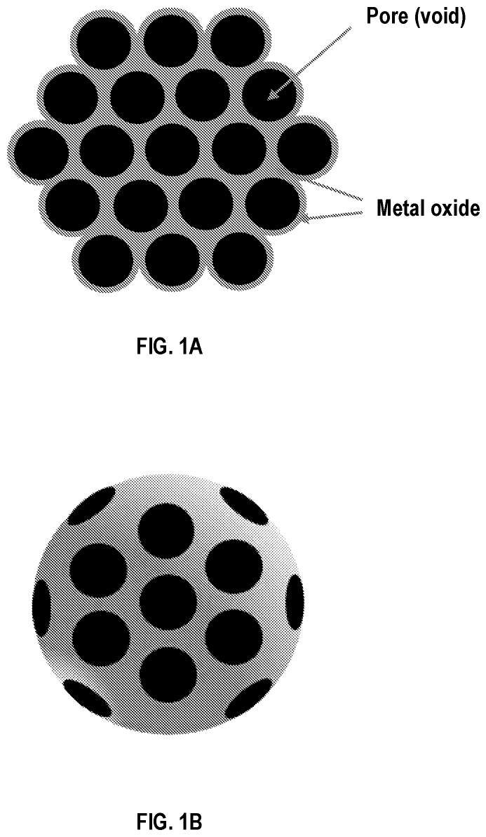

Embodiments of the present disclosure are directed to closed-cell metal oxide particles comprising a metal oxide matrix having an array of pores (referred to as “void volumes” or “voids,” which may comprise air) formed therein of substantially uniform sizes, as illustrated by a cross-sectional view in FIG. 1A. As illustrated, the closed-cell metal oxide particle is formed from a metal oxide matrix which defines an array of “closed-cells” that encapsulate media-inaccessible void volumes. An outer surface of the closed-cell metal oxide particle (depicted as an overcoated surface formed by the metal oxide) is defined by the array of closed-cells such that there are substantially no open pores of similar size to the closed-cells at the surface.

In contrast to the present embodiments, the porous metal oxide particle shown in FIG. 1B has pores on its exterior surface and connected pores inside. When formulated into a medium, the medium infiltrates into these pores, resulting in a loss of color effects in the downstream formulation due to the refractive index match between medium and matrix material. This greatly limits the applications of porous particles in a variety of formulations. The closed-cell metal oxide particles of the present embodiments are impermeable to polymers and large molecules frequently used in such formulations, and thus can prevent penetration into the pores and retaining air in the pores. Thus, the close-cell metal oxide particles advantageously maintain a constant net refractive index between the matrix and voids regardless of the surrounding media in the application.

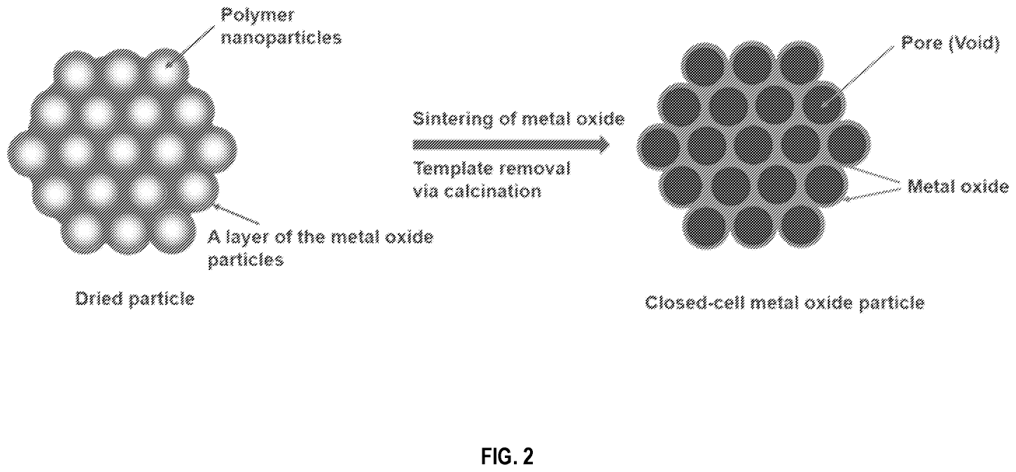

FIG. 2 illustrates an exemplary process for forming the closed-cell metal oxide particles. In certain embodiments, the closed-cell metal oxide particles are produced by drying droplets of a formulation comprising a matrix of metal oxide particles on the order of 1 to 120 nm in diameter, and polymer particles on the order of 50 to 500 nm which will serve as the template. In certain embodiments, the two particle species are oppositely charged (e.g., positively charged polymer particles and negatively charged metal oxide particles) to facilitate formation of a coating of the metal oxide particles on the polymer particles. In certain embodiments, a spray drying or microfluidics process is used to generate the droplets (e.g., aqueous droplets), and the droplets are dried to remove their solvent. In certain embodiments that utilize a spray drying process, the generation of droplets and drying is performed in rapid succession. During the drying process, the polymer particles and the metal oxide particles self-assemble to form a microsphere containing polymer particles embedded in a metal oxide matrix. By sintering the matrix nanoparticles, for example, in a muffle furnace, the matrix nanoparticles densify and form a stable matrix around the polymer particles. During this process, the polymer particles are removed via calcination, resulting in a final closed-cell particle having an array of closed-cells formed therein.

The resulting closed-cell metal oxide particles may be micron-scaled, for example, having average diameters from about 0.5 μm to about 100 μm. In certain embodiments, the closed-cell metal oxide particles have an average diameter from about 0.5 μm, about 0.6 μm, about 0.7 μm, about 0.8 μm, about 0.9 μm, about 1.0 μm, about 5.0 μm, about 10 μm, about 20 μm, about 30 μm, about 40 μm, about 50 μm, about 60 μm, about 70 μm, about 80 μm, about 90 μm, about 100 μm, or within any range defined by any of these average diameters (e.g., about 1.0 μm to about 20 μm, about 5.0 μm to about 50 μm, etc.). The metal oxide employed may also be in particle form, and may be nano-scaled. The metal oxide matrix particles may have an average diameter, for example, of about 1 nm to about 120 nm. The polymer template particles may have an average diameter, for example, of about 50 nm to about 500 nm. One or more of the polymer particles or the metal oxide particles may be polydisperse or monodisperse. In certain embodiments, the metal oxide may be provided as metal oxide particles or may be formed from a metal oxide precursor, for example, via a sol-gel technique.

Certain embodiments of the closed-cell metal oxide particles exhibit color in the visible spectrum at a wavelength range selected from the group consisting of 380 nm to 450 nm, 451 nm to 495 nm, 496 nm to 570 nm, 571 nm to 590 nm, 591 nm to 620 nm, 621 nm to 750 nm, 751 nm to 800 nm, and any range defined therebetween (e.g., 496 nm to 620 nm, 450 nm to 750 nm, etc.). In some embodiments, the particles exhibit a wavelength range in the ultraviolet spectrum selected from the group consisting of 100 nm to 400 nm, 100 nm to 200 nm, 200 nm to 300 nm, and 300 nm to 400 nm.

In certain embodiments, the closed-cell metal oxide particles can have, for example, one or more of an average diameter of from about 0.5 μm to about 100 μm, an average porosity of greater than about 0.1, greater than about 0.2, greater than about 0.3, greater than about 0.4, greater than about 0.5, greater than about 0.6, greater than about 0.7, greater than about 0.8, or about 0.10 to about 0.80, and an average pore diameter of from about 50 nm to about 500 nm. In other embodiments, the particles can have, for example, one or more of an average diameter of from about 1 μm, to about 75 μm, an average porosity of from about 0.10 to about 0.40, and an average pore diameter of from about 50 nm to about 800 nm.

In certain embodiments, the closed-cell metal oxide particles have an average diameter, for example, of from about 1 μm to about 75 μm, from about 2 μm to about 70 μm, from about 3 μm to about 65 μm, from about 4 μm to about 60 μm, from about 5 μm to about 55 μm, or from about 5 μm to about 50 μm; for example, from any of about 5 μm, about 6 μm, about 7 μm, about 8 μm, about 9 μm, about 10 μm, about 11 μm, about 12 μm, about 13 μm, about 14 μm, or about 15 μm to any of about 16 μm, about 17 μm, about 18 μm, about 19 μm, about 20 μm, about 21 μm, about 22 μm, about 23 μm, about 24 μm, or about 25 μm. Other embodiments can have an average diameter of from any of about 4.5 μm, about 4.8 μm, about 5.1 μm, about 5.4 μm, about 5.7 μm, about 6.0 μm, about 6.3 μm, about 6.6 μm, about 6.9 μm, about 7.2 μm, or about 7.5 μm to any of about 7.8 μm about 8.1 μm, about 8.4 μm, about 8.7 μm, about 9.0 μm, about 9.3 μm, about 9.6 μm, or about 9.9 μm.

In certain embodiments, the closed-cell metal oxide particles have an average porosity, for example, of from any of about 0.10, about 0.12, about 0.14, about 0.16, about 0.18, about 0.20, about 0.22, about 0.24, about 0.26, about 0.28, about 0.30, about 0.32, about 0.34, about 0.36, about 0.38, about 0.40, about 0.42, about 0.44, about 0.46, about 0.48 about 0.50, about 0.52, about 0.54, about 0.56, about 0.58, or about 0.60 to any of about 0.62, about 0.64, about 0.66, about 0.68, about 0.70, about 0.72, about 0.74, about 0.76, about 0.78, about 0.80, or about 0.90. Other embodiments can have an average porosity of from any of about 0.45, about 0.47, about 0.49, about 0.51, about 0.53, about 0.55, or about 0.57 to any of about 0.59, about 0.61, about 0.63, or about 0.65.

In some embodiments, the closed-cell metal oxide particles have an average pore diameter of about 3 nm, about 4 nm, about 5 nm, about 10 nm, about 20 nm, or about 25 nm to about 30 nm, about 35 nm, about 40 nm, about 45 nm, or about 50 nm. In other embodiments, the metal oxide particles have an average pore diameter, for example, of from any of about 10 nm, about 20 nm, about 30 nm, about 40 nm, about 50 nm, about 60 nm, about 70 nm, about 80 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, or about 440 nm to any of about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, or about 800 nm. Other embodiments can have an average pore diameter of from any of about 220 nm, about 225 nm, about 230 nm, about 235 nm, about 240 nm, about 245 nm, or about 250 nm to any of about 255 nm, about 260 nm, about 265 nm, about 270 nm, about 275 nm, about 280 nm, about 285 nm, about 290 nm, about 295 nm, or about 300 nm.

In certain embodiments, the metal oxide material of the closed-cell metal oxide particles is selected from silica, titania, alumina, zirconia, ceria, iron oxides, zinc oxide, indium oxide, tin oxide, chromium oxide, or combinations thereof. In certain embodiments, the metal oxide comprises titania, silica, or a combination thereof.

In certain embodiments, the polymer of the polymer particles is selected from poly(meth)acrylic acid, poly(meth)acrylates, polystyrenes, polyacrylamides, polyvinyl alcohol, polyvinyl acetate, polyesters, polyurethanes, polyethylene, polypropylene, polylactic acid, polyacrylonitrile, polyvinyl ethers, derivatives thereof, salts thereof, copolymers thereof, or combinations thereof. For example, the polymer is selected from the group consisting of polymethyl methacrylate, polyethyl methacrylate, poly(n-butyl methacrylate), polystyrene, poly(chloro-styrene), poly(alpha-methylstyrene), poly(N-methylolacrylamide), styrene/methyl methacrylate copolymer, polyalkylated acrylate, polyhydroxyl acrylate, polyamino acrylate, polycyanoacrylate, polyfluorinated acrylate, poly(N-methylolacrylamide), polyacrylic acid, polymethacrylic acid, methyl methacrylate/ethyl acrylate/acrylic acid copolymer, styrene/methyl methacrylate/acrylic acid copolymer, polyvinyl acetate, polyvinylpyrrolidone, polyvinylcaprolactone, polyvinylcaprolactam, a co-polymer of methyl methacrylate and [2-(methacryloyloxy)ethyl]trimethylammonium chloride, derivatives thereof, salts thereof, or combinations thereof.

In certain embodiments, a weight to weight ratio of the metal oxide particles to the polymer particles is from about 1/10, about 2/10, about 3/10, about 4/10, about 5/10 about 6/10, about 7/10, about 8/10, about 9/10, to about 10/9, about 10/8, about 10/7, about 10/6, about 10/5, about 10/4, about 10/3, about 10/2, or about 10/1. In certain embodiments, the weight to weight ratio of the metal oxide particles to the polymer particles is 1/3, 2/3, 1/1, or 3/2.

In further embodiments, the closed-cell metal oxide particles can have, e.g., from about 60.0 wt % to about 99.9 wt % metal oxide, based on the total weight of the closed-cell metal oxide particles. In other embodiments, the closed-cell metal oxide particles comprise from about 0.1 wt % to about 40.0 wt % of one or more light absorbers, based on the total weight of the closed-cell metal oxide particles. In other embodiments, the metal oxide is from any of about 60.0 wt %, about 64.0 wt %, about 67.0 wt %, about 70.0 wt %, about 73.0 wt %, about 76.0 wt %, about 79.0 wt %, about 82.0 wt % or about 85.0 wt % to any of about 88.0 wt %, about 91.0 wt %, about 94.0 wt %, about 97.0 wt %, about 98.0 wt %, about 99.0 wt % or about 99.9 wt % metal oxide, based on the total weight of the closed-cell metal oxide particles.

In certain embodiments, the closed-cell metal oxide particles are prepared by a process comprising forming a liquid dispersion of polymer particles and metal oxide particles; forming liquid droplets of the dispersion; drying the liquid droplets to provide polymer template particles comprising polymer and metal oxide; and removing the polymer to provide closed-cell metal oxide particles. In such embodiments, the resulting closed-cells (and thus the encapsulated voids) are monodisperse.

In certain embodiments, the closed-cell metal oxide particles are prepared by a method comprising: generating liquid droplets from a particle dispersion comprising metal oxide particles and polymer particles; drying the liquid droplets to provide dried particles comprising a matrix of the metal oxide particles embedded with the polymer particles; and calcining or sintering the dried particles to densify the metal oxide particle matrix and remove the polymer particles, resulting in closed-cell metal oxide particles.

In other embodiments, the closed-cell metal oxide particles are prepared by a process comprising: generating liquid droplets from a particle dispersion comprising polymer particles and a sol-gel of a metal oxide; drying the liquid droplets to provide dried particles comprising a matrix of the metal oxide with the polymer particles; and calcining or sintering the dried particles to remove the polymer particles, resulting in closed-cell metal oxide particles. An exemplary process is described as follows: liquid droplets are generated from a particle dispersion (e.g., an aqueous particle dispersion with a pH of 3-5) comprising polymer particles and a precursor of a metal oxide. The precursor may be, for example, tetraethyl orthosilicate (TEOS) or tetramethyl orthosilicate (TMOS) as a silica precursor, titanium propoxide as a titania precursor, or zirconium acetate as a zirconium precursor. The liquid droplets are dried to provide dried particles comprising a hydrolyzed precursor of metal oxide that surrounds and coats the polymer particles. The dried particles are then heated to sinter the metal oxide via a condensation reaction of the hydrolyzed precursor, and to remove the polymer particles via calcination.

In some embodiments, the evaporation of the liquid medium may be performed in the presence of self-assembly substrates such as conical tubes or silicon wafers. In certain embodiments, dried particle mixtures may be recovered, e.g., by filtration or centrifugation. In some embodiments, the drying comprises microwave irradiation, oven drying, drying under vacuum, drying in the presence of a desiccant, or a combination thereof.

In certain embodiments, droplet formation and collection occur within a microfluidic device. Microfluidic devices are, for example, narrow channel devices having a micron-scaled droplet junction adapted to produce uniform size droplets, with the channels being connected to a collection reservoir. Microfluidic devices, for example, contain a droplet junction having a channel width of from about 10 μm to about 100 μm. The devices are, for example, made of polydimethylsiloxane (PDMS) and may be fabricated, for example, via soft lithography. An emulsion may be prepared within the device via pumping an aqueous dispersed phase and oil continuous phase at specified rates to the device where mixing occurs to provide emulsion droplets. Alternatively, an oil-in-water emulsion may be utilized. The continuous oil phase comprises, for example, an organic solvent, a silicone oil, or a fluorinated oil. As used herein, “oil” refers to an organic phase (e.g., an organic solvent) immiscible with water. Organic solvents include hydrocarbons, for example, heptane, hexane, toluene, xylene, and the like.

In certain embodiments with liquid droplets, the droplets are formed with a microfluidic device. The microfluidic device can contain a droplet junction having a channel width, for example, of from any of about 10 μm, about 15 μm, about 20 μm, about 25 μm, about 30 μm, about 35 μm, about 40 μm, or about 45 μm to any of about 50 μm, about 55 μm, about 60 μm, about 65 μm, about 70 μm, about 75 μm, about 80 μm, about 85 μm, about 90 μm, about 95 μm, or about 100 μm.

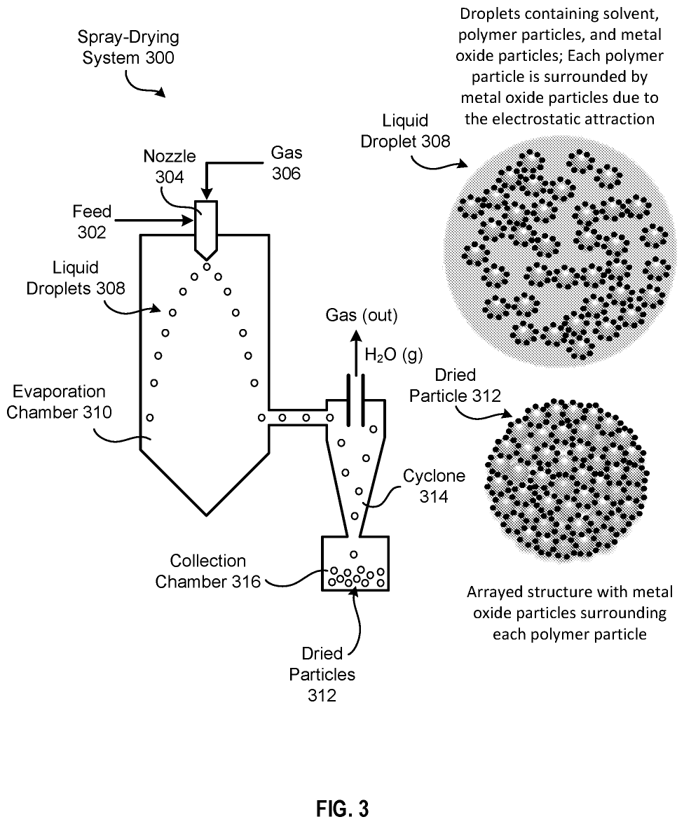

In certain embodiments, generating and drying the liquid droplets is performed using a spray-drying process. FIG. 3 shows a schematic of an exemplary spray drying system 300 used in accordance with various embodiments of the present disclosure. In certain embodiments of spray-drying techniques, a feed 302 of a liquid solution or dispersion is fed (e.g. pumped) to an atomizing nozzle 304 associated with a compressed gas inlet through which a gas 306 is injected. The feed 302 is pumped through the atomizing nozzle 304 to form liquid droplets 308. The liquid droplets 308 are surrounded by a pre-heated gas in an evaporation chamber 310, resulting in evaporation of solvent to produce dried particles 312. The dried particles 312 are carried by the drying gas through a cyclone 314 and deposited in a collection chamber 316. Gases include nitrogen and/or air. In an embodiment of an exemplary spray-drying process, a liquid feed contains a water or oil phase, the metal oxide, and the polymer particles. The dried particles 312 comprise a self-assembled structure of each polymer particle surrounded by metal oxide particles.

Air may be considered a continuous phase with a dispersed liquid phase (a liquid-in-gas emulsion). In certain embodiments, spray-drying comprises an inlet temperature of from any of about 100° C., about 105° C., about 110° C., about 115° C., about 120° C., about 130° C., about 140° C., about 150° C., about 160° C., or about 170° C. to any of about 180° C., about 190° C., about 200° C., about 210° C., about 215° C., or about 220° C. In some embodiments a pump rate (feed flow rate) of from any of about 1 mL/min, about 2 mL/min, about 5 mL/min, about 6 mL/min, about 8 mL/min, about 10 mL/min, about 12 mL/min, about 14 mL/min, or about 16 mL/min to any of about 18 mL/min, about 20 mL/min, about 22 mL/min, about 24 mL/min, about 26 mL/min, about 28 mL/min, or about 30 mL/min is utilized.

In some embodiments, vibrating nozzle techniques may be employed. In such techniques, a liquid dispersion is prepared, and then droplets are formed and dropped into a bath of a continuous phase. The droplets are then dried. Vibrating nozzle equipment is available from BÜCHI and comprises, for example, a syringe pump and a pulsation unit. Vibrating nozzle equipment may also comprise a pressure regulation valve.

In certain embodiments, polymer removal may be performed, for example, via calcination, pyrolysis, or with a solvent (solvent removal). Calcination is performed in some embodiments at temperatures of at least about 200° C., at least about 500° C., at least about 1000° C., from about 200° C. to about 1200° C., or from about 200° C. to about 700° C. The calcining can be for a suitable period, e.g., from about 0.1 hour to about 12 hours or from about 1 hour to about 8.0 hours. In other embodiments, the calcining can be for at least about 0.1 hour, at least about 1 hour, at least about 5 hours, or at least about 10 hours. In other embodiments, the calcining can be from any of about 200° C., about 350° C., about 400° C., 450° C., about 500° C. or about 550° C. to any of about 600° C., about 650° C., about 700° C., or about 1200° C. for a period of from any of about 0.1 h (hour), about 1 h, about 1.5 h, about 2.0 h, about 2.5 h, about 3.0 h, about 3.5 h, or about 4.0 h to any of about 4.5 h, about 5.0 h, about 5.5 h, about 6.0 h, about 6.5 h, about 7.0 h, about 7.5 h about 8.0 h, or about 12 h. While the polymer is removed during this process, an array of void volumes will be substantially maintained by the closed-cells left behind after the calcination.

In certain embodiments, a particle size ratio of the metal oxide particles to the polymer particles is from 1/50 to 1/5 (e.g., 1/10).

In certain embodiments, the metal oxide particles have an average diameter of from about 1 nm, about 5 nm, about 10 nm, about 15 nm, about 20 nm, about 25 nm, about 30 nm, about 35 nm, about 40 nm, about 45 nm, about 50 nm, about 55 nm, or about 60 nm to about 65 nm, about 70 nm, about 75 nm, about 80 nm, about 85 nm, about 90 nm, about 95 nm, about 100 nm, about 105 nm, about 110 nm, about 115 nm, or about 120 nm. In other embodiments, the matrix nanoparticles have an average diameter of about 5 nm to about 150 nm, about 50 to about 150 nm, or about 100 to about 150 nm.

In certain embodiments, the polymer particles have an average diameter of from about 50 nm to about 990 nm. In other embodiments, the particles have an average diameter of from any of about 50 nm, about 75 nm, about 100 nm, about 130 nm, about 160 nm, about 190 nm, about 210 nm, about 240 nm, about 270 nm, about 300 nm, about 330 nm, about 360 nm, about 390 nm, about 410 nm, about 440 nm, about 470 nm, about 500 nm, about 530 nm, about 560 nm, about 590 nm, or about 620 nm to any of about 650 nm, about 680 nm, about 710 nm, about 740 nm, about 770 nm, about 800 nm, about 830 nm, about 860 nm, about 890 nm, about 910 nm, about 940 nm, about 970 nm, or about 990 nm.

In certain embodiments, removing the polymer particles comprises calcination, pyrolysis, or solvent removal. The calcining of the polymer particles can be, e.g., at temperatures of from about 300° C. to about 800° C. for a period of from about 1 hour to about 8 hours.

In certain embodiments, the closed-cell metal oxide particles comprise mainly metal oxide, that is, they may consist essentially of or consist of metal oxide. Advantageously, depending on the particle compositions, relative sizes, and shapes of the metal oxide particles used, a bulk sample of the closed-cell metal oxide particles may exhibit color observable by the human eye, may appear white, or may exhibit properties in the UV spectrum. A light absorber may also be present in the particles, which may provide a more saturated observable color. Absorbers include inorganic and organic materials, for example, a broadband absorber such as carbon black. Absorbers may, for example, be added by physically mixing the particles and the absorbers together or by including the absorbers in the droplets to be dried. In certain embodiments, a closed-cell metal oxide particle may exhibit no observable color without added light absorber and exhibit observable color with added light absorber.

The closed-cell metal oxide particles described herein may exhibit angle-dependent color or angle-independent color. “Angle-dependent” color means that observed color has dependence on the angle of incident light on a sample or on the angle between the observer and the sample. “Angle-independent” color means that observed color has substantially no dependence on the angle of incident light on a sample or on the angle between the observer and the sample.

Angle-dependent color may be achieved, for example, with the use of monodisperse polymer particles. Angle-dependent color may also be achieved when a step of drying the liquid droplets is performed slowly, allowing the particles to become ordered. Angle-independent color may be achieved when a step of drying the liquid droplets is performed quickly, not allowing the particles to become ordered.

The following embodiments may be utilized to achieve angle-dependent color resulting from ordered pores left behind after polymer removal. As a first example embodiment of angle-dependent color, monodisperse and spherical polymer particles are embedded in metal oxide particles, and the metal oxide particles are subsequently densified and the polymer is removed. The metal oxide particles may be spherical or non-spherical. As a second example embodiment of angle-dependent color, two or more species of polymer particles that are collectively monodisperse and spherical are embedded in metal oxide particles, and the metal oxide particles are subsequently densified and the polymer is removed. Angle-dependent color is achieved independently of the polydispersity and shapes of the matrix particles.

The following embodiments may be utilized to achieve angle-independent color resulting from disordered pores left behind after polymer removal. As a first example embodiment of angle-independent color, polydisperse polymer particles are embedded in metal oxide particles, and the metal oxide particles are subsequently densified and the polymer is removed.

As a second example embodiment of angle-independent color, two different sized polymer particles (i.e., a bimodal distribution of monodisperse polymer particles) are embedded in metal oxide particles, and the metal oxide particles are subsequently densified and the polymer is removed. The metal oxide particles may be spherical or non-spherical.

As a third example embodiment of angle-independent color, two different sized and polydisperse spherical polymer particles are embedded in metal oxide particles, and the metal oxide particles are subsequently densified and the polymer is removed.

Angle-independent color is achieved independently of the polydispersity and shapes of the matrix particles.

Any of the embodiments exhibiting angle-dependent or angle-independent color may be modified to exhibit whiteness or effects (e.g., reflectance, absorbance) in the ultraviolet spectrum.

In some embodiments, the metal oxide particles can comprise combinations of different types of particles. For example, the metal oxide particles may be a mixture of two different metal oxides (i.e., discrete distributions of metal oxide particles), such as a mixture of alumina particles and silica particles with each species being characterized by the same or similar size distributions.

In some embodiments, the metal oxide particles may comprise more complex compositions and/or morphologies. For example, the metal oxide particles may comprise particles such that each individual particle comprises two or more metal oxides (e.g., silica-titania particles). Such particles may comprise, for example, a mixture of two or more metal oxides.

In some embodiments, the metal oxide particles and/or the polymer particles may comprise surface functionalization. An example of a surface functionalization is a silane coupling agent (e.g., silane-functionalized silica). In some embodiments, the surface functionalization is performed on the metal oxide particles prior to self-assembly and densification. In some embodiments, the surface functionalization is performed on the closed-cell metal oxide particles after densification. In some embodiments, the surface-functionalization may be selected to impart a net positive or net negative surface charge to the particles when dispersed in an aqueous solution.

Particle size, as used herein, is synonymous with particle diameter and is determined, for example, by scanning electron microscopy (SEM) or transmission electron microscopy (TEM). Average particle size is synonymous with D50, meaning half of the population resides above this point, and the other half resides below this point. Particle size refers to primary particles. Particle size may be measured by laser light scattering techniques with dispersions or dry powders.

Mercury porosimetry analysis can be used to characterize the porosity of the particles. Mercury porosimetry applies controlled pressure to a sample immersed in mercury. External pressure is applied for the mercury to penetrate into the voids/pores of the material. The amount of pressure required to intrude into the voids/pores is inversely proportional to the size of the voids/pores. A mercury porosimeter generates volume and pore size distributions from the pressure versus intrusion data generated by the instrument using the Washburn equation. Porosity, as reported herein for closed-cell metal oxide particles, is calculated as a ratio of unoccupied space and total particle volume. For example, porous silica particles containing voids/pores with an average size of 165 nm have an average porosity of 0.8.

### ILLUSTRATIVE EXAMPLES

The following examples are set forth to assist in understanding the disclosed embodiments and should not be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

### Example 1: Preparation of Closed-Cell Silica Particles Via Microfluidic Technology

An aqueous dispersion of positively charged poly(meth)acrylate nanoparticle was diluted to 1 wt % with deionized water and 3 wt % of negatively charged silica nanoparticles was added. The mixture was sonicated for 30 seconds to prevent agglomeration. The aqueous nanoparticle dispersion and oil phase (a continuous oil phase containing 2 wt % of polyethylene glycol-co-perfluoro polyester surfactant in fluorinated oil) were each injected into a microfluidic device having a 50 μm droplet junction via syringe pumps. The system was allowed to equilibrate until monodispersed droplets were produced. The droplets were collected in a reservoir.

Collected droplets were dried in an oven at 50° C. for 4 hours. The dried powder was calcined by placing on a silicon wafer, heating from room temperature to 500° C. over a 4 hour period, holding at 500° C. for 2 hours, and cooling back to room temperature over a 4 hour period. The procedure resulted in monodispersed closed-cell silica particles having a diameter of 15 micrometers.

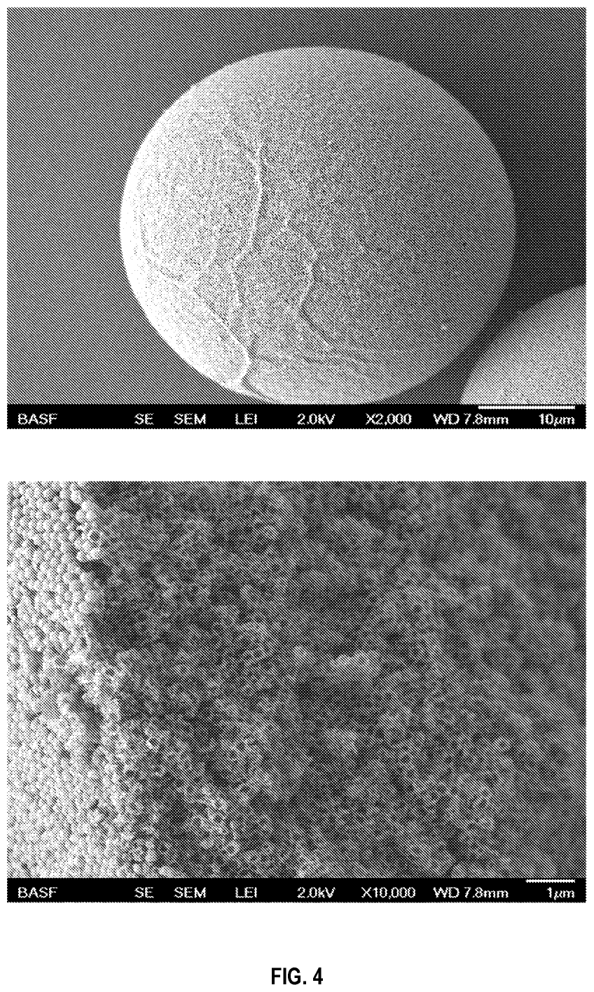

FIG. 4 shows SEM images of a closed-cell metal oxide particle produced according to a microfluidics process (top image), as well as a cross-section of a closed-cell metal oxide particle (bottom image) revealing that the interior structure comprise an array of closed-cell metal oxide shells that each encompass relatively monodisperse and ordered voids.

### Example 2: Closed-Cell Silica Particles Encapsulating Media-Inaccessible Void Volume

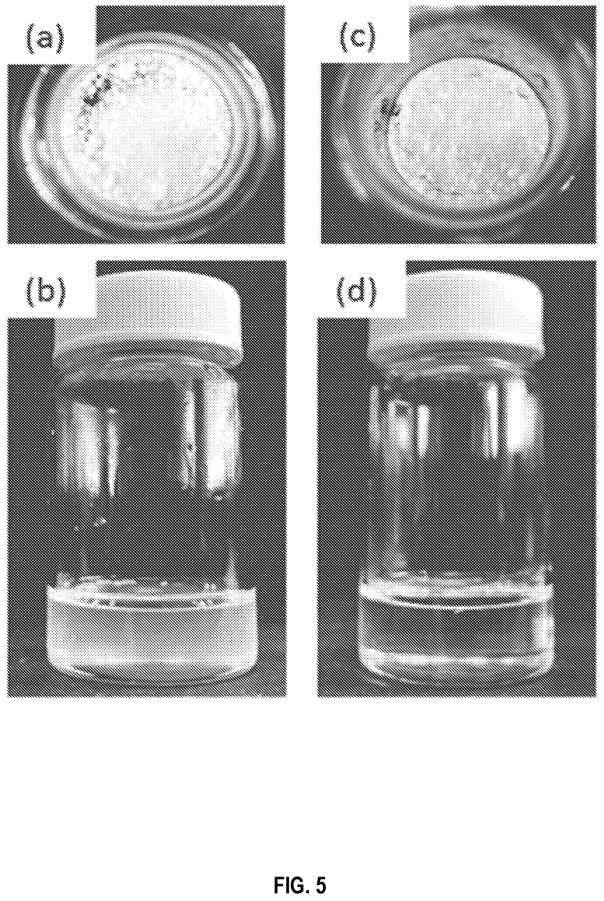

The powder product from Example 1 is dispersed in mineral oil at a mass concentration of 3 wt %. The same concentration of porous silica particles was also dispersed in mineral oil for comparison. FIG. 5 shows photographs of (a) the powder product of closed-cell silica particles, (b) the closed-cell silica particles in mineral oil, (c) the powder product of porous silica particles, and (d) the porous silica particles in mineral oil. The suspension of closed-cell silica particles exhibited a cloudy appearance. The closed-cell silica particles do not disappear in mineral oil, which has a refractive index of 1.46-1.47, demonstrating that the closed-cell morphology can prevent medium from infiltrating into the enclosed voids. In comparison, the suspension of porous silica particles exhibited a clear appearance. The porous particles disappear after the oil infiltrates the voids due to the refractive index match between the silica (which has a refractive index of about 1.47) and the mineral oil.

### Example 3: Closed-Cell Silica Particles with Ordered Voids Produced Via Spray-Drying

An aqueous suspension of positively charged spherical polymer nanoparticles (co-polymer of methyl methacrylate and 2-(methacryloyloxy)ethyl]trimethylammonium chloride nanoparticles having an average diameter of 254 nm) and negatively charged silica nanoparticles (having an average diameter of 7 nm) was prepared. The polymer nanoparticles were present at 1.8 wt % and the silica nanoparticles were present at 0.6 wt % based on a weight of the aqueous suspension (a 3:1 weight to weight ratio of polymer nanoparticles to metal oxide nanoparticles). The aqueous suspension was spray dried under an inert atmosphere (nitrogen) at a 100° C. inlet temperature, a 40 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to sinter and densify the silica nanoparticles and remove the polymer to produce the closed-cell silica particles. The heating parameters were as follows: the particles were heated from room temperature to 550° C. over a period of 5 hours, held at 550° C. for 2 hours, and then cooled back to room temperature over a period of 3 hours.

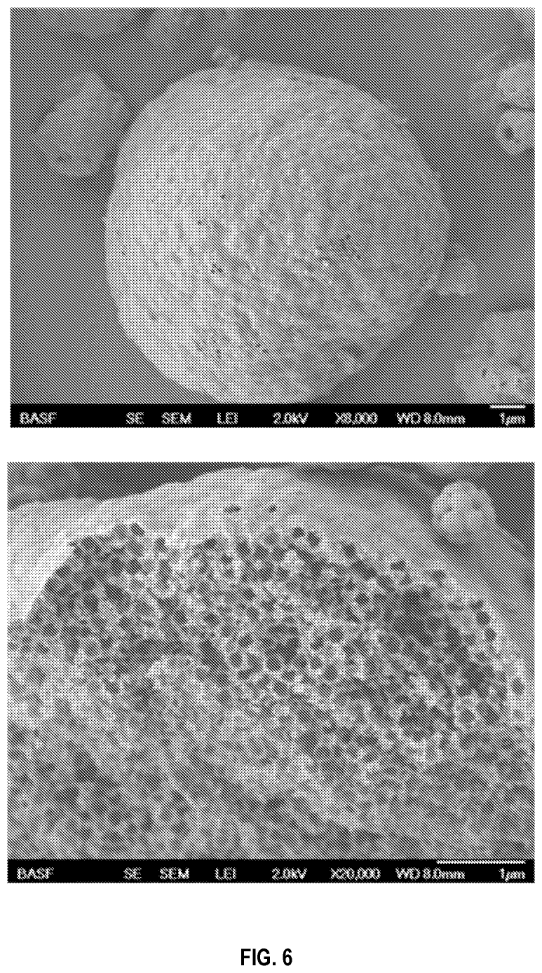

FIG. 6 shows SEM images of a closed-cell silica particle produced according to a spray drying process (left image), as well as a cross-section of a closed-cell silica particle (right image) revealing that the interior structure comprise an array of closed-cell silica shells that each encompass relatively monodisperse and ordered voids.

### Example 4: Closed-Cell Silica Particles Containing a Light Absorber

The product of Example 1 was physically mixed with an aqueous dispersion of carbon black or a carbon black powder at varying weight levels. The resulting closed-cell silica particles contained carbon black at levels of 0.5 wt. %, 1 wt. %, 2 wt. %, 3 wt. %, 4 wt. % and 5 wt. %, based on the total weight of the particles.

### Example 5: Visible Color in Bulk Samples

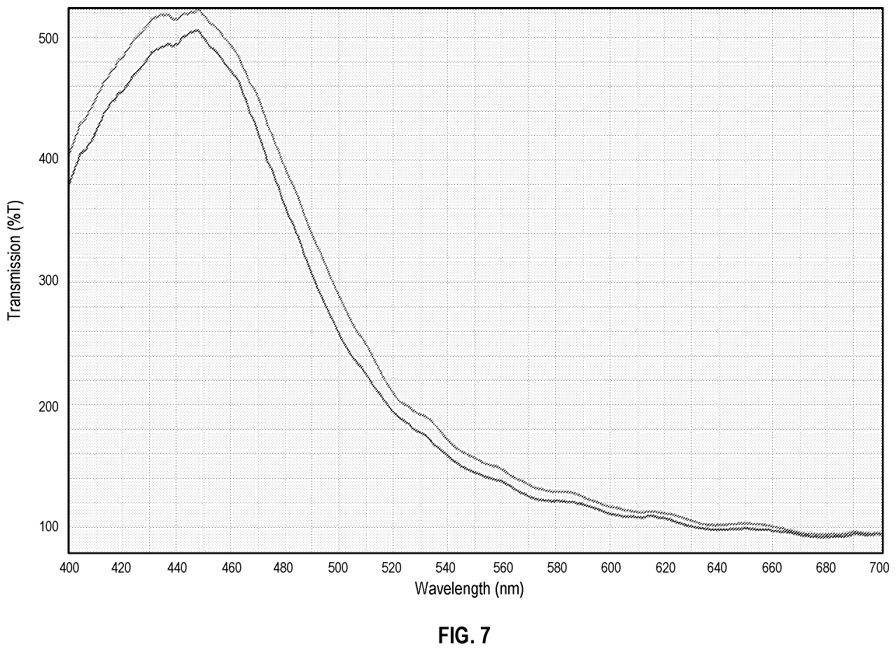

The closed-cell silica particles of Example 1 (0.5 mg) were evenly distributed in a 20-mL clear glass vial having a 6 cm² bottom surface. The sample exhibited a distinct blue color that was observable by the human eye. FIG. 7 is a plot of the UV-vis spectrum for this sample, which shows a reflection peak at 440 nm corresponding to blue color.

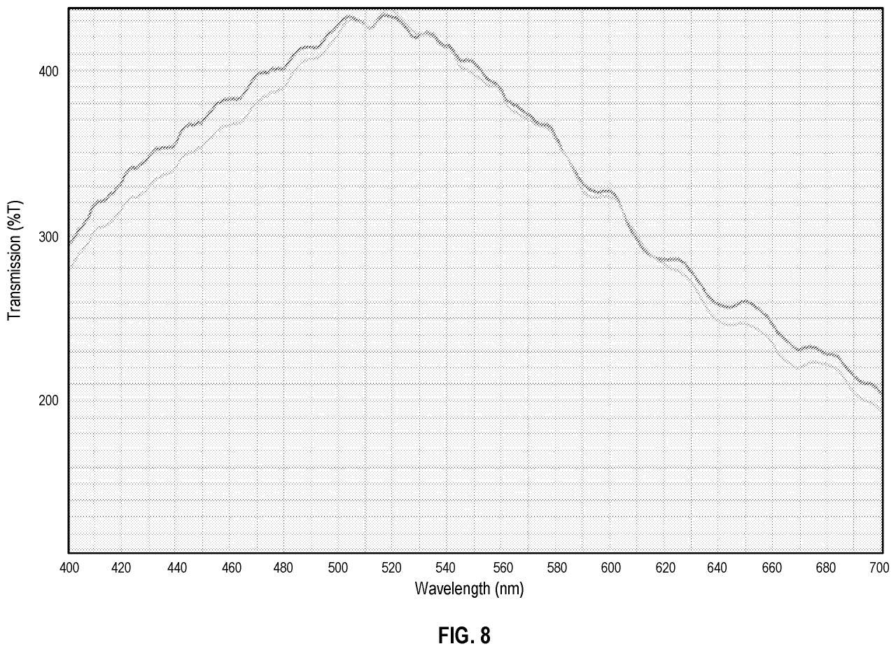

A sample of closed-cell silica particles was produced in a similar fashion to Example 1, except the weight to weight ratio of polymer to silica was 2:1. The sample exhibited distinct green color observable by the human eye. FIG. 8 is a plot of the UV-vis spectrum for this sample, which shows a reflection peak at 520 nm corresponding to green color.

### Example 6: Closed-Cell Silica Particles Demonstrating UV Attenuation

A sample of closed-cell silica particles was produced in a similar fashion to Example 1, except that PMMA nanoparticles having a diameter of 140 nm were used, and the weight to weight ratio of polymer to silica was 3:1. The sample exhibited attenuation in the UV range. Closed-cell silica particles showed attenuation in UV range. The UV attenuation of silica nanoparticles were used as a control sample and the relative low attenuation value suggested that the UV attenuation of closed-cell silica particles did not come from the silica nanoparticles.

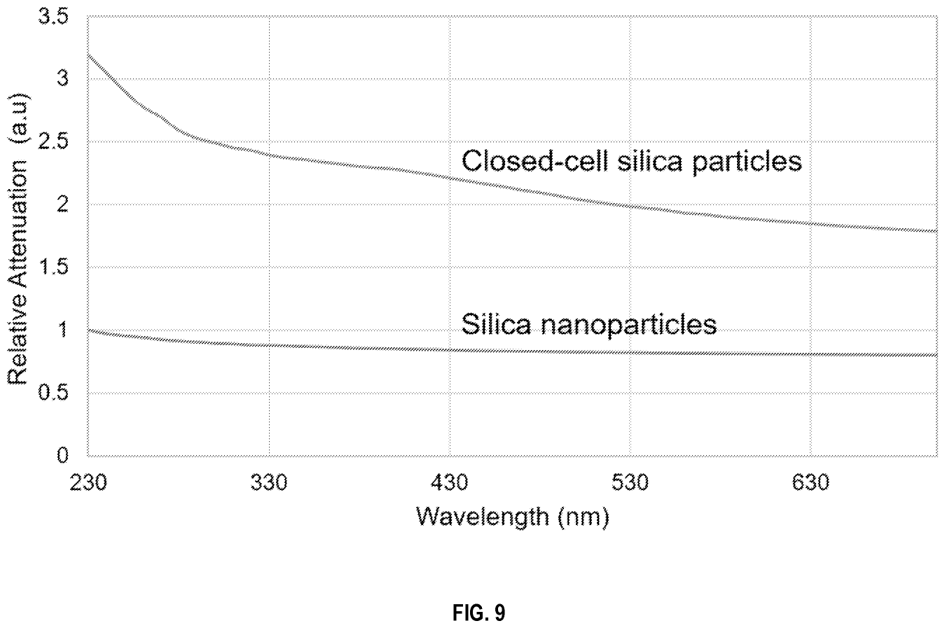

FIG. 9 is a plot showing relative attenuation values of closed-cell silica particles and silica nanoparticles in UV range.

### Example 7: Closed-Cell Titania Particles

An aqueous suspension of negatively charged spherical polystyrene nanoparticles (having an average diameter of 197 nm) and positively charged titania nanoparticles (having an average diameter of 15 nm) was prepared. The polymer nanoparticles were present at 1.8 wt. % and the titania nanoparticles were present at 1.2 wt. % based on a weight of the aqueous suspension (a 3:2 weight to weight ratio of polymer nanoparticles to metal oxide nanoparticles). The aqueous suspension was spray dried under an inert atmosphere (nitrogen) at a 100° C. inlet temperature, a 55 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to sinter and densify the titania nanoparticles and remove the polymer to produce the closed-cell titania particles. The heating parameters were as follows: the particles were heated from room temperature to 300° C. over a period of 4 hours, held at 300° C. for 6 hours, and then heated to 550° C. over a period of 2 hours, held at 550° C. for 2 hours, and cooled back to room temperature over a period of 4 hours.

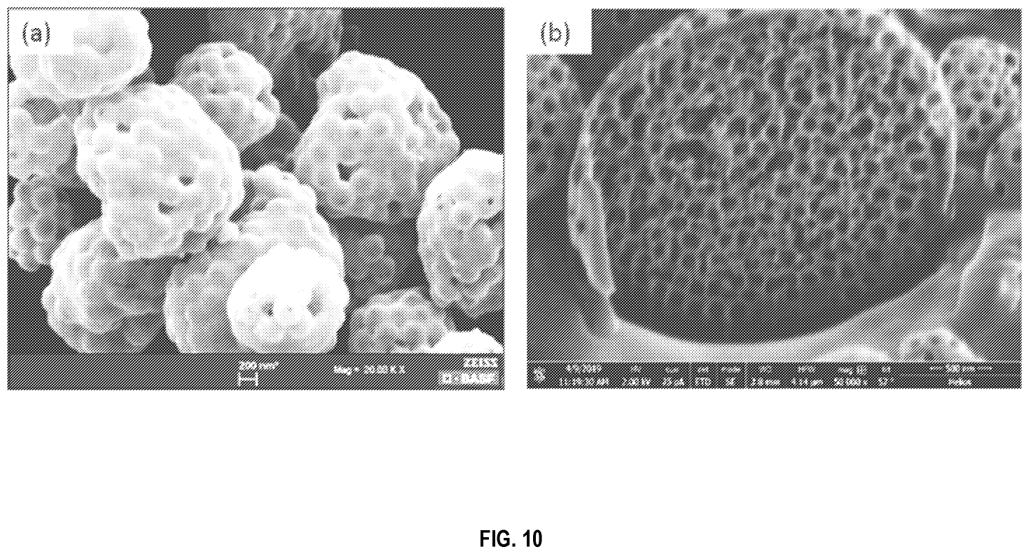

FIG. 10 shows SEM images of a closed-cell titania particle produced according to a spray drying process (left image), as well as a cross-section of a closed-cell titania particle (right image) revealing that the interior structure comprises an array of closed-cell titania shells that each encompass relatively monodisperse voids.

### Example 8: Preparation of Closed-Cell Silica Particle Via Sol-Gel Process

An aqueous suspension of positively charged spherical polymer nanoparticles (co-polymer of methyl methacrylate and 2-(methacryloyloxy)ethyl trimethylammonium chloride nanoparticles having an average diameter of 254 nm) and silica precursor tetramethyl orthosilicate (TMOS) was mixed in the pH range of 2-5. The polymer nanoparticles were present at 1.8 wt. % and the TMOS were present at 3.6 wt. % based on a weight of the aqueous suspension (a 1:3 weight to weight ratio of polymer nanoparticles to metal oxide). The aqueous suspension was spray dried under an inert atmosphere (nitrogen) at a 100° C. inlet temperature, a 40 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to convert silica precursor to silica nanoparticles and densify the silica, and remove the polymer to produce closed-cell silica particles. The heating parameters were as follows: the particles were heated from room temperature to 200° C. over a period of 3 hours, held at 200° C. for 2 hours, and then heated to 550° C. over a period of 2 hours, held at 550° C. for 2 hours and cooled back to room temperature over a period of 3 hours.

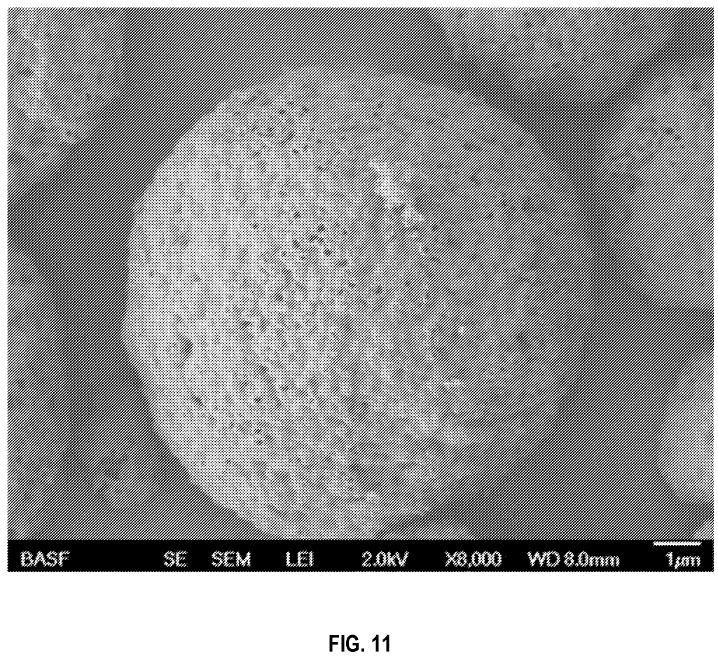

FIG. 11 shows an SEM image of the product produced in Example 8.

### Example 9: Closed-Cell Silica Particles with Disordered Voids

An aqueous suspension of two different sized (254 nm and 142 nm in diameter, respectively) positively charged spherical polymer nanoparticles (co-polymer of methyl methacrylate and 2-(methacryloyloxy)ethyl trimethylammonium chloride nanoparticles) and negatively charged silica nanoparticles (having an average diameter of 7 nm) was prepared. The polymer nanoparticles were present at 1.8 wt. % in total (0.9 wt. % of each) and the silica nanoparticles were present at 0.6 wt. % based on a weight of the aqueous suspension. The aqueous suspension was spray dried under an inert atmosphere (nitrogen) at a 100° C. inlet temperature, a 40 mm spray gas pressure, a 100% aspirator rate, and a 30% flow rate (about 10 mL/min) using a BÜCHI lab-scale spray dryer.

The spray dried powder was removed from the spray dryer's collection chamber and spread onto a silicon wafer for sintering. The spray dried powder was then calcined in a muffle furnace with a batch sintering process to convert silica precursor to silica nanoparticles and densify the silica, and remove the polymer to produce closed-cell silica particles. The heating parameters were as follows: the particles were heated from room temperature to 550° C. over a period of 6 hours, held at 550° C. for 2 hours, and then cooled back to room temperature over a period of 4 hours.

The closed-cell silica particles (0.5 mg) were evenly distributed in a 20-mL clear glass vial having a 6 cm² bottom surface. The sample exhibited an angle-independent blue color that was observable by the human eye.

In the foregoing description, numerous specific details are set forth, such as specific materials, dimensions, processes parameters, etc., to provide a thorough understanding of the embodiments of the present disclosure. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

Reference throughout this specification to “an embodiment,” “certain embodiments,” or “one embodiment” means that a particular feature, structure, or characteristic described in connection with the embodiment is included in at least one embodiment. Thus, the appearances of the phrase “an embodiment,” “certain embodiments,” or “one embodiment,” in various places throughout this specification are not necessarily all referring to the same embodiment, and such references mean “at least one.”

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

1. A method of preparing closed-cell metal oxide particles, the method comprising:
generating liquid droplets from a particle dispersion comprising first particles comprising a polymer material and second particles comprising a metal oxide material;
drying the liquid droplets to provide dried particles comprising an array of the first particles, wherein each of the first particles is coated by a layer of the second particles; and
calcining or sintering the dried particles, wherein the calcining or sintering densifies the metal oxide material and removes the polymer material to produce the closed-cell metal oxide particles each comprising a metal oxide matrix defining an array of closed-cells, each closed-cell encapsulating a media-inaccessible void volume, and wherein outer surfaces of the closed-cell metal oxide particles are defined by their respective arrays of closed-cells.

2-23. (canceled)

24. Closed-cell metal oxide particles prepared by a process of claim 1.

25. A closed-cell metal oxide particle comprising a metal oxide matrix defining an array of closed-cells, each closed-cell encapsulating a media-inaccessible void volume, wherein the outer surface of the closed-cell metal oxide particle is defined by the array of closed-cells.

26. The closed-cell metal oxide particle of claim 25, wherein the array of closed-cells is an ordered array.

27. The closed-cell metal oxide particle of claim 25, wherein the array of closed-cells is a disordered array.

28. The closed-cell metal oxide particle of claim 25, wherein the void volumes have an average diameter from about 50 nm to about 500 nm.

29. The closed-cell metal oxide particle of claim 25, wherein the metal oxide matrix comprises a metal oxide selected from silica, titania, alumina, zirconia, ceria, iron oxides, zinc oxide, indium oxide, tin oxide, chromium oxide, and combinations thereof.

30. The closed-cell metal oxide particle of claim 25, wherein the metal oxide matrix comprises silica.

31. The closed-cell metal oxide particle of claim 25, derived at least partially from polymer particles having an average diameter from about 50 nm to about 500 nm.

32. The closed-cell metal oxide particle of claim 25, derived at least partially from metal oxide particles having an average diameter from about 1 nm to about 120 nm.

33. The closed-cell metal oxide particle of claim 25, derived from a metal oxide precursor selected from silica, titania, alumina, zirconia, ceria, iron oxides, zinc oxide, indium oxide, tin oxide, chromium oxide, and combinations thereof.

34. A composition comprising a plurality of the closed-cell metal oxide particle of claim 25.

35. The composition of claim 34, wherein an average diameter of the closed-cell metal oxide particles range from about 0.5 μm to about 100 μm.

36. The composition of claim 34, further comprising a substrate having the closed-cell metal oxide particles disposed thereon.

37. The composition of claim 34, wherein the composition is an aqueous formulation, an oil-based formulation, an ink, a coating formulation, a food, a plastic, a cosmetic formulation or a material for a medical application, or a security application.

38. A bulk composition exhibiting whiteness, a non-white color, or an effect in the ultraviolet spectrum, the bulk composition comprising a plurality of the closed-cell metal oxide particles of claim 24.

39. The composition of claim 34, further comprising a light absorber.

40. The composition of claim 39, wherein the light absorber is present from 0.1 wt % to about 40.0 wt %.

41. The composition of claim 39, wherein the light absorber comprises carbon black.

42. The composition of claim 39, wherein the light absorber comprises one or more ionic species.
